(19) United States
(12) Patent Application Publication
(10) Pub. No.: US 2024/0430539 A1
(43) Pub. Date: Dec. 26, 2024

(54) METHOD AND AUDIOVISUAL SET-TOP BOX FOR RECOMMENDING A LIST OF AT LEAST ONE AUDIOVISUAL PROGRAMME

(71) Applicant: SAGEMCOM BROADBAND SAS, Bois-Colombes (FR)

(72) Inventors: Piero INDIVERI, Bois-Colombes (FR); Vincent SCHOTT, Bois-Colombes (FR); Jérôme BERGER, Bois-Colombes (FR)

(73) Assignee: SAGEMCOM BROADBAND SAS, Bois-Colombes (FR)

(21) Appl. No.: 18/746,867

(22) Filed: Jun. 18, 2024

(30) Foreign Application Priority Data
Jun. 26, 2023 (FR) 2306657

(51) Int. Cl. H04N 21/482; A61B 5/16

(52) U.S. Cl. CPC H04N 21/4826; A61B 5/165

(57) ABSTRACT

A method and an audiovisual set-top box for recommending a list of at least one audiovisual programme to at least one user, the programme or programmes being categorised in genres and/or subgenres. The method has steps, performed by the audiovisual decoder, of: associating said genres and/or said subgenres with emotional states; detecting a current emotional state of said at least one user; performing a sorting of said programmes in said catalogue taking account at least of said emotional state detected and said genres and/or subgenres associated with said emotional state detected; and displaying a list of at least one audiovisual programme taking account of said sorting of said programmes in said catalogue. This makes it possible to take account of the detected current emotional state of the user in order to improve the relevance of the programmes that are recommended to them.

### TECHNICAL FIELD

The technical field of the invention is that of broadcasting audiovisual contents or programmes, in particular. The present invention relates more particularly to a method for recommending an audiovisual programme or list of audiovisual programmes taking account of the emotion of the user.

### PRIOR ART

Platforms for distributing programmes (in particular audiovisual ones) have available more and more extensive catalogues of programmes. It is therefore sometimes difficult for the user to seek and select a programme that suits them. To facilitate this selection, platforms generally use methods for recommending programmes taking account in particular of the category of programme and of the number of readings of these programmes so as to offer to the user the most popular programmes in each category.

However, this technique is not satisfactory since it does not take account of the current emotional state of the user and the programmes that are offered to them are therefore not necessarily in phase with the expectations of the user at this moment. This is because, depending on the emotional state of the user, their preferences may differ and may not correspond to the recommendations conventionally made by the distribution platforms. There is therefore a need to provide a method for recommending a programme, in particular audiovisual, to a user taking account in particular of their emotional state in order to improve the relevance of the programmes that are offered to them.

### DISCLOSURE OF THE INVENTION

For this purpose, according to a first aspect, the invention proposes a method for recommending a list of at least one audiovisual programme from a catalogue of programmes to at least one user oriented to a set-top box, in particular an audiovisual set-top box, and display means, said programme is in said catalogue being categorised in genres and/or subgenres.

Catalogue, throughout this description, means a programme base available for reading by a user, here via an audiovisual set-top box.

It should be noted that the present invention is described in particular in relation to reading audiovisual programmes. It will easily be understood that the present solution could be implemented for recommending programmes of the audio and/or visual type, such as podcasts, audiobooks, music, etc, or photographs, in particular.

Thus, reading means the fact that a user, during a predetermined period, maintains the reproduction, by the audiovisual set-top box 10, of a programme, whatever the type of programme.

For reasons of clarity and understanding of the invention, it is described hereinafter that the programmes in the catalogue are categorised by genre. It is understood here that a genre (for example film/drama) can comprise a plurality of subgenres (for example

Adventure/western/war, Science fiction/fantasy/horror, Comedy, etc) or that the genre can in fact, in an alternative, be a subgenre.

The invention also relates to an audiovisual set-top box able to implement such a recommendation method.

Thus, the present invention makes it possible to take account of the detected current emotional state of at least one user in order to improve the relevance of the list of at least one programme that is recommended to them.

According to a particular aspect of the invention, the method comprises the steps performed by said audiovisual set-top box of:
associating said genres with emotional states;
detecting a current emotional state of said at least one user;
implementing a sorting of said programmes in said catalogue of programmes taking account at least of said emotional state detected of said genres associated with said emotional state detected;
displaying a list of at least one audiovisual programme taking account of said sorting of said programmes in said catalogue of programmes.

According to a particular embodiment of the invention, said step of associating said genres with said emotional states comprises a substep of using a priority table wherein, for each of said emotional states, said genres are classified in an order of priority, said classification taking account at least of a counter of readings of each genre associated with each emotional state, said priority table furthermore comprising a default genre attributed last according to the order of priority for each of said emotional states.

According to a particular aspect of this particular embodiment, said step of implementing a sorting of said programmes in said catalogue taking account of said emotional state detected and of said genres associated with said emotional state detected comprises the substeps of:
pointing, in said priority table, to said genre classified first according to said order of priority for said emotional state detected, and
detecting a selection by the user and, if the user does not select a programme corresponding to said first genre pointed to, pointing to the following genre in the priority table until the user selects a programme in said genre pointed to or until said genre pointed to is said default genre.

According to a particular aspect of this particular embodiment, said step of displaying said list of at least one audiovisual programme taking account of said sorting of said programmes in said catalogue comprises the substeps of:
ordering said at least one programme in said list of at least one programme to be displayed, said ordering at least partly taking account of a priority criterion of said at least one programme; and
displaying said list of at least one programme according to said ordering.

According to another particular embodiment of the invention, said step of associating said genres with said emotional states comprises a substep of using a counting table comprising a counter of readings by the user for each genre associated with an emotional state.

According to a particular aspect of this other particular embodiment, said step of implementing a sorting of said programmes in said catalogue taking account at least of said emotional state detected and of said genres associated with said emotional state detected comprises the substeps of:
calculating a weighting coefficient for each of said genres associated with said emotional state detected;
for each programme included in a list of programmes offered in which each programme has a predetermined score and a genre, calculating a weighted score by multiplying said predetermined score of said programme by said weighting coefficient corresponding to said genre;
ranking said programmes in said list of programmes offered by decreasing order of weighted score.

According to a particular aspect of this other particular embodiment, the substep of calculating a weighting coefficient for each of said genres associated with said emotional state detected comprises:
for said emotional state detected, determining a maximum value among said counters associated with each of said genres;
for each genre associated with said emotional state detected, dividing said value of said corresponding counter by said maximum value determined.

According to a another particular aspect of this other particular embodiment, said step of implementing a sorting of said programmes in said catalogue taking account at least of said emotional state detected and of said genres associated with said emotional state detected comprises the substeps of:
ranking the programmes in a list of programmes offered, wherein each programme has a predetermined score, by decreasing order of said counter associated with said genre and with said emotional state detected; then
ranking said programmes in said list of programmes having the same genre by decreasing order of the predetermined score.

According to a particular aspect of the invention, the method comprises a step of updating said priority table or said counting table, when said at least one user reads, during at least one predetermined period, one of said recommended programmes, by incrementing a value of said counter associated with said programme read by said at least one user.

According to another particular aspect of the invention, each genre comprises at least one subgenre and said priority table and said counting table comprise a counter of readings for each genre and a counter of readings for each subgenre. The step of updating said priority table and said counting table comprising the substeps of, when said at least one user reads one of said recommended programmes:
incrementing said counters of the genre and of the subgenre of the programme selected by said at least one user corresponding to said emotional state detected by a first value;
incrementing the counters of the other subgenres associated with the genre of the programme selected by said at least one user corresponding to said emotional state detected by a second value.

According to a particular aspect, when said audiovisual set-top box detects at least two users, the method comprises:
a step of determining the main user on the basis of at least one predetermined criterion, said substep of using a priority table or a counting table being performed using said priority table or said counting table associated with said main user determined; or
a step of combining said priority or counting tables of each of said users detected, said substep of using a priority table or a counting table being performed using the priority table or counting table resulting from this combination.

According to a particular aspect of the invention, the method comprises a step of triggering said method:
when said audiovisual set-top box is switched on; or
when at least one user is detected by said audiovisual set-top box; or
when a programme is selected/sought by said at least one user.

The invention also proposes an audiovisual set-top box for implementing the method for recommending an audiovisual programme or a list of audiovisual programmes from a catalogue of programmes to at least one user as described previously, said programmes in said catalogue being categorised in genres. Said audiovisual set-top box being intended to be connected to a remote database, a display means and a control device that can be used by a user for controlling said audiovisual set-top box, said set-top box comprising electronic circuitry adapted and configured for:
associating said genres with emotional states;
detecting a current emotional state of said at least one user;
performing a sorting of said programmes in said catalogue taking account at least of said emotional state detected of said genres associated with said emotional state detected;
said display means being intended to display a list of at least one audiovisual programme taking account of said sorting of said programmes in said catalogue.

A computer program, which can be stored on a medium and/or downloaded from a communication network, in order to be read by a processor, is also proposed here. This computer program comprises instructions for implementing the method mentioned above in any one of the embodiments thereof, when said program is executed by a processor. The invention also relates to an information storage medium storing such a computer program.

### BRIEF DESCRIPTION OF THE DRAWINGS

The features of the invention mentioned above, as well as others, will emerge more clearly from the reading of the following description of an example embodiment, said description being made in relation to the accompanying drawings, among which:

FIG. 1 illustrates schematically a telecommunication system in which the present invention is implemented;

FIG. 2 illustrates schematically an example of hardware architecture of an audiovisual set-top box of the invention;

FIG. 3 illustrates schematically a recommendation method according to the invention;

FIG. 4 illustrates schematically a step of associating genres with emotional states of the method of FIG. 3, according to a first embodiment;

FIG. 5 shows an example of a priority table used in the method of FIG. 3, the priority table being in a first state;

FIG. 6 illustrates schematically a step of performing a sorting of the programmes in a catalogue taking account at least of an emotional state detected and of genres associated with the emotional state detected of the method of FIG. 3, according to a first embodiment;

FIG. 7 shows an example of a priority table when used in the method of FIG. 3, the priority table being in a second state;

FIG. 8 shows an example of a priority table when used in the method of FIG. 3, the priority table being in an initial state;

FIG. 9 illustrates schematically a step of displaying at list of at least one audiovisual programme taking account of the sorting of the programmes in the catalogue of the method of FIG. 3;

FIG. 10 illustrates schematically a method of filling in the priority table by learning, according to the first embodiment;

FIG. 11 illustrates schematically a step of associating genres with emotional states of the method of FIG. 3, according to a second embodiment;

FIG. 12 shows an example of a counting table used in the method of FIG. 3, the counting table being in an initial state;

FIG. 13 illustrates schematically a step of performing a sorting of the programmes in a catalogue taking account at least of an emotional state detected and of genres associated with the emotional state detected of the method of FIG. 3, according to the second embodiment;

FIG. 14 shows an example of a list of programmes offered by the operator, each programme comprising a predetermined score;

FIG. 15 shows an example of a counting table used in the method of FIG. 3, wherein the counters have been incremented following the implementation of the method of FIG. 3;

FIG. 16 illustrates an example of a table for calculating a weighted score;

FIG. 17 illustrates an example of a weighting table of a list of programmes offered by the operator;

FIG. 18 illustrates an example of a list of programmes ranked by implementing the method of FIG. 3, according to the second embodiment;

FIG. 19 illustrates schematically a step of updating the counting table, according to the second embodiment;

FIG. 20 illustrates schematically a variant of a step of performing a sorting of the programmes in a catalogue taking account at least of an emotional state detected and of genres associated with the emotional state detected of the method of FIG. 3, according to the second embodiment;

FIG. 21 illustrates schematically a variant of a step of updating the priority table or the counting table, compatible with the first and second embodiments; and

FIG. 22 illustrates schematically a step of detecting the number of users of the method of FIG. 3.

### DETAILED DISCLOSURE OF EMBODIMENTS

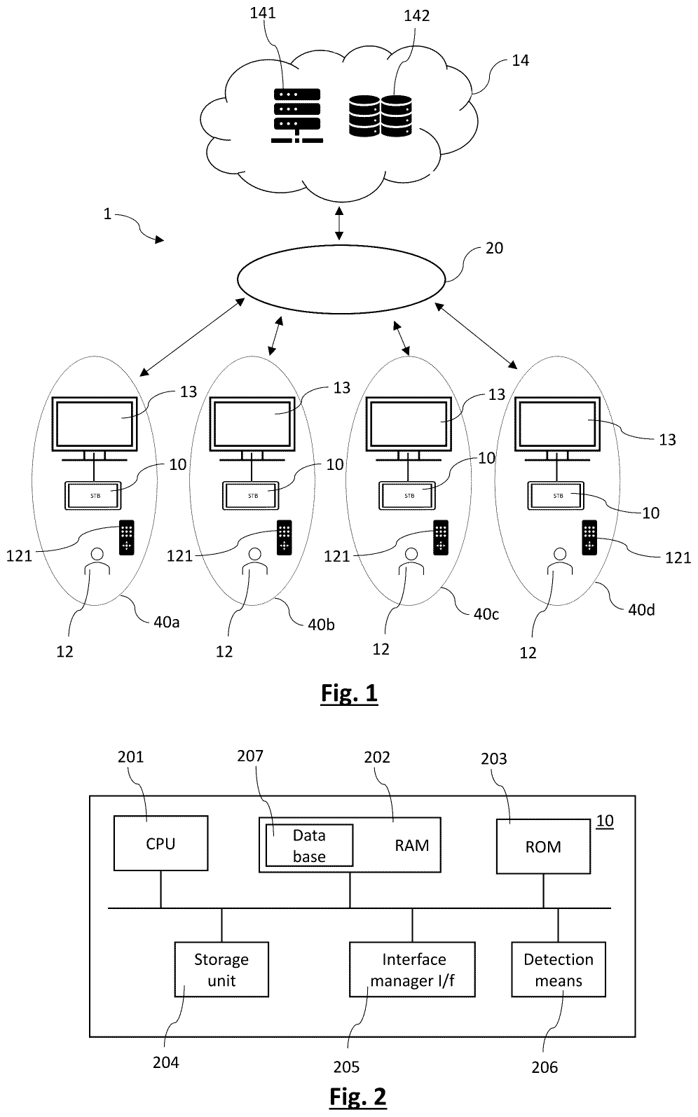

FIG. 1 illustrates schematically a telecommunication system 1 in which the present invention is implemented.

In FIG. 1, an external data source 14 comprising a server 141 and a remote database 142 is connected by means of a wide area network 20 to local area networks (LANs) 40. The wide area network 20 is for example a network of the internet type. The remote database 142 is for example configured to store the information relating to the audiovisual programmes.

For example, the local area networks 40 are wired and/or wireless domestic networks. Only four domestic networks 40a-40d are shown in FIG. 1 for reasons of simplification. In a particular example, the local area networks 40a-40d are wireless networks of the Wi-Fi type.

In the example in FIG. 1, each local area network 40a-40d comprises an audiovisual set-top box 10 for implementing a method for recommending at least one list of programmes. The set-top box 10 is connected to at least one display means 13 (by an HDMI (High Definition Multimedia Interface) link or SCART (Syndicat des Constructeurs d′Appareils Radiorécepteurs et Téléviseurs) link, or any other type of adapted link) and to a control device 121 (preferably via a wireless connection) used by at least one user 12 for controlling the audiovisual set-top box 10. According to one approach, the user can interact with the audiovisual set-top box 10 via a microphone integrated in the audiovisual set-top box 10. In the example illustrated, a single user and a single display means are shown.

FIG. 2 illustrates schematically an example of hardware architecture for implementing, in the form of electronic circuitry, the audiovisual set-top box according to the invention. According to the example of hardware architecture shown in FIG. 2, the set-top box 10 comprises, connected by a communication bus 200: a processor or CPU (“central processing unit”) 201; a random access memory (RAM) 202; a read only memory 203, for example of the ROM (“read-only memory”) or EEPROM (“electrically-erasable programmable read-only memory”) type, such as a flash memory; a storage unit 204, such as a hard disk drive HDD or a storage medium reader, such as an SD (Secure Digital) card reader; at least one interface manager I/f 205. The interface manager I/f 205 enables the audiovisual set-top box 10 to communicate by means of the wide area network 20 and to interact with the control device 121.

The processor 201 is capable of executing instructions loaded in the RAM 202 from the ROM 203, from an external memory (not shown), from a storage medium (such as an SD card), or from a communication network. When the audiovisual set-top box 10 is powered up, the processor 201 is capable of reading instructions from the RAM 202 and executing them. These instructions form a computer program causing the implementation, by the processor 201, of all or part of the method described here.

The storage unit 204, or the random access memory 202, is able to store a local database 207 storing the priority and/or counting tables described in more detail later in this description.

The set-top box 10 furthermore comprises means 206 for detecting the emotion of a user. To do this, the detection means 206 comprise video and/or audio capture means (for example at least one camera and/or a microphone) of the user as well as an association module configured to associate the captured images and/or audio with a current emotional state of the user. Optionally, the detection means 206 may use a connected device of the IoT (standing for “Internet of Things”) type based on biomarkers.

All or part of the method and steps described above can thus be implemented in software form by executing a set of instructions by a programmable machine, for example a processor of the DSP (“digital signal processor”) type, or a microcontroller, or be implemented in hardware form by a machine or a dedicated electronic component (chip) or a dedicated set of electronic components (chipset), for example an FPGA (field-programmable gate array) or an ASIC (application-specific integrated circuit). In general terms, the audiovisual set-top box 10 comprises electronic circuitry adapted and configured to implement the method described here.

The method described below in relation to FIGS. 3 to 22 can be implemented in software form by executing a set of instructions by a programmable machine, for example a DSP (“digital signal processor”), or a microcontroller, or be implemented in hardware form by a machine or a dedicated component, for example an FPGA (field-programmable gate array) or an ASIC (application-specific integrated circuit). In general, the audiovisual set-top box 10 comprises electronic circuitry configured for implementing the method described in relation to FIGS. 3 to 22.

It should be noted here that FIG. 2 shows a hardware architecture of a single audiovisual set-top box 10. Some of the various elements constituting the audiovisual set-top box 10 can be distributed in various computer devices included in a computing cloud. FIG. 3 illustrates schematically a method 100 for recommending a list of at least one audiovisual programme from a catalogue of audiovisual programmes according to the present invention. The recommendation method 100 is intended for at least one user 12 oriented to the audiovisual set-top box 10 and to the display means 13. It should be noted that the programmes in the programme catalogue are categorised in genres and, according to another approach, in genres and subgenres. The information relating to the category of each programme can be recovered by the audiovisual set-top box 10 in accordance with known methods. For example, broadcasting a programme over satellite, cable or terrestrial television networks uses the DVB (Digital Video Broadcasting) standard, which provides for the signalling of metadata relating to a programme via the EIT table (“Event Information Table”). An EIT table can in particular indicate the genre of the programme via the description of the content, as detailed in the ETSI document EN 300 468. The audiovisual set-top box 10 of the invention is therefore able to obtain the information relating to the genre of the programmes by an adapted means (of the IP (Internet Protocol), DSL (Digital Subscriber Line), cable, satellite, etc connection type) from the external data source 14, which may be a satellite, cable, DTT (digital terrestrial television or IP source or any other audiovisual stream source.

According to another approach, the information relating to the programmes may be recovered by the audiovisual set-top box 10 via a description text file (JSON (JavaScript Object Notation) for example) downloaded from an internet server. In general, the possible programme genres used according to the invention are those given by the content provider (DVB, OTT (Over-The-Top), etc).

For example, the programmes may be categorised according to the following genres: Film/Drama, News, Televised show/game, Sports, Programme for children/young persons, Music/Ballet/Dance, Arts/Culture (without music), Social/political/economic questions, Education/science/factual subjects, Leisure, etc.

Optionally, the programmes may furthermore be categorised in subgenres, such as for example for the Film/drama genre: General, Detective/thriller, Adventure/western/war, Science fiction/fantasy/horror, Comedy, Serial/melodrama/folklore, Romance, Serious/classical/religious/historical film/drama, Adult film/drama.

It will obviously be understood that the invention can use all or some of the genres listed above. The invention can also use other genres not listed above. In addition, the invention can use the main genres alone or in combination with their subgenres.

Moreover, the audiovisual set-top box 10 has available a database 207 containing a certain number of emotional states already detected or that it is capable of detecting (as described below) by the detection means 206. These emotional states are for example included in: anger, disgust, fear, joy, sadness, surprise, neutrality, etc. It will obviously be understood that the invention can use all or some of the emotional states listed above. The invention can also use other emotional states not listed above.

According to the invention, the recommendation method 100 comprises the steps, performed by the audiovisual decoder 10, of:
associating E20 the genres with emotional states;
detecting E40 a current emotional state of the user;
performing a sorting E60 of said programmes in said catalogue taking account at least of said emotional state detected and said genres associated with said emotional state detected;
displaying E80 a list of at least one audiovisual programme taking account of said sorting of said programmes in said catalogue.

In order to facilitate understanding of the method according to the first embodiment, in describing it account is taken only of the programme genres (and not of the subgenres). It will easily be understood that the method of the invention could take account, in addition to or in place of the genres, of the programme subgenres without departing from the operating principle of this embodiment. One example embodiment taking account of subgenres is in particular described later in the description.

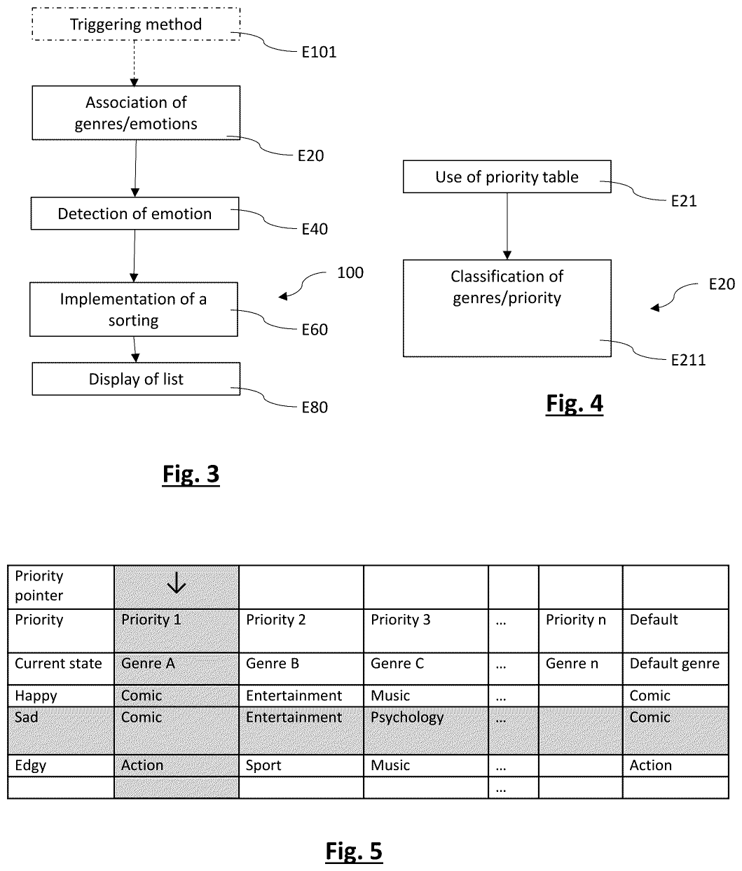

According to a first embodiment, the step E20 of associating the genres with emotional states comprises, as illustrated on FIG. 4, a substep E21 of using a priority table. More precisely, the priority table comprises a list of common emotional states with which offers of programme genres are associated. The priority table makes possible to classify E211 in an order of priority each genre offered for each emotional state. This classifying E211 of the genres associated with each emotional state takes account at least of a counter of readings, by the user, of each genre associated with each emotional state. In other words, for each emotional state, the genres are classified by order of priority, i.e., for a given emotional state, the genres are classified by order of the genres most read by the user when the user is in the given emotional state. The counters of readings locally count the number of readings by the user, for example in the home.

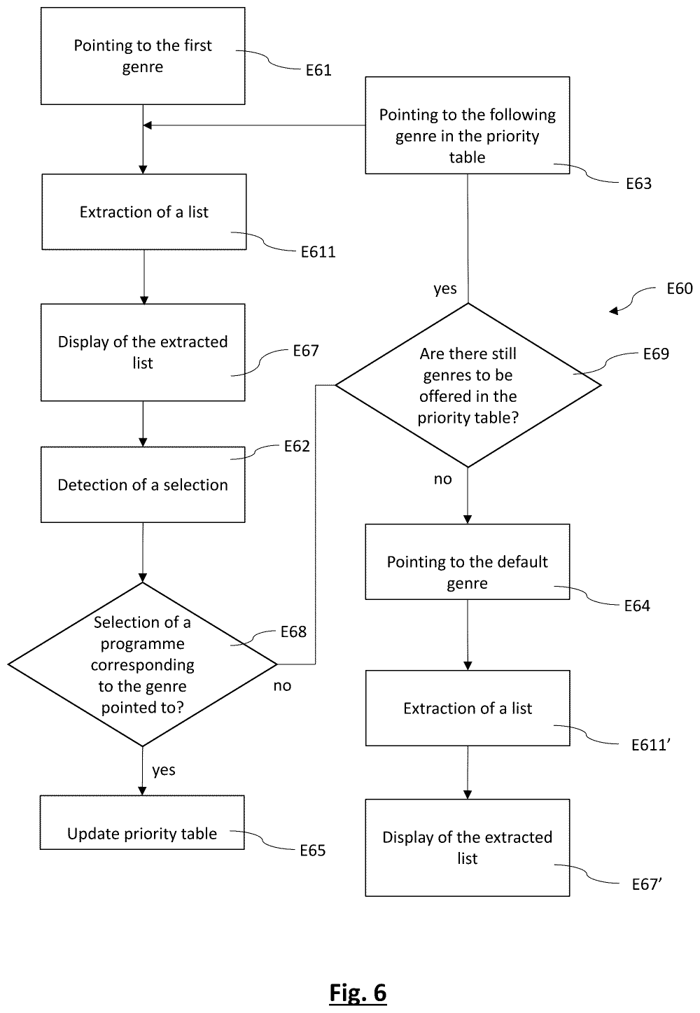

For example, and as illustrated on FIG. 5, which shows a priority table according to the first embodiment, for the emotional state corresponding to sadness, the genre classified first in the order of priority is the comic genre, then the entertainment genre and then the psychology genre. This means that, when the user is in the emotional state corresponding to sadness, they have watched more programmes in the comic genre, then the entertainment genre and then the psychology genre.

As aforementioned, classifying the genres by emotional state takes account in particular of a counter of readings associated with each genre for each emotional state. Other criteria can however be taken into account to classify the genres associated with each emotional state. In addition, it can also be envisaged predefining the order of preference of the genres to be offered for each emotional state. This predefined order could for example be configured by the user when the audiovisual set-top box 10 is configured or by the operator supplying the audiovisual set-top box 10.

It should be noted that, in the priority table shown on FIG. 5, only three genres are detailed but n genres could be associated with each emotional state. Quite obviously it will be understood that a greater or lesser number than three genres could be associated with each emotional state. The number of genres associated with each emotional state could also be limited, for example to 20 genres per emotional state, so as to limit the size of the priority table and to optimise the storage space in particular.

Optionally, the number of genres taken into account in the priority table (i.e. the number of priorities) could correspond to the total number of genres offered by the broadcasting standard of the audiovisual contents. Optionally, the number of genres taken into account in the priority table could be extended, for example by the broadcaster or by the audiovisual set-top box 10 when the addition of a new genre is detected. This addition can for example take place at the end of the table with the addition of a new column in the priority table.

In addition, a default genre (and optionally a default subgenre) is provided for each emotional state and is offered to the user when the priority table is initialised or when the user does not select any of the genres offered, as described hereinafter.

The priority table can be embedded in the audiovisual set-top box 10 or downloaded from an external network. For example, one possible implementation of the priority table can be done in SQL (Structured Query Language) or this may be a file of the JSON type.

The step E40 of detecting a current emotional state of the user 12 can for example use a known detection technique that consists of using neural networks trained to detect the emotions of an individual from images. This technique can for example use, for extracting the characteristics of the face of the individual from the image: algorithms of the Gabor filter, Histogram of Oriented Gradients (HOG) or a Local Binary Pattern (LBP) type. For the classification, this technique can for example use a support vector machine (SVM), a random forest (RF) or a K-nearest neighbour (KNN) algorithm.

Other techniques make it possible to detect the emotional state of an individual from their voice, as described in the documents U.S. Pat. No. 11,341,986 or TW201606760A.

According to the first embodiment, the step of performing a sorting E60 of the programmes in the catalogue taking account at least of the emotional state detected and the genres (and/or subgenres when the method takes account of subgenres) associated with the emotional state detected makes it possible to extract a programme or a list of programmes to be offered/recommended to the user according to their current emotional state detected.

To do this, the audiovisual set-top box 10 refers to the priority table in FIG. 5. The audiovisual set-top box 10 performs the step of performing a sorting E60 that comprises the substeps of (illustrated on FIG. 6):
pointing E61, in said priority table, to the genre classified first according to the order of priority for the emotional state detected, and
detecting E62 a selection by the user and, if the user does not select a programme corresponding to said first genre pointed to, pointing E63 to the following genre in the priority table until the user selects a programme in said genre pointed to or until said genre pointed to is said default genre E64.

More precisely, and referring to the priority table illustrated on FIG. 5, when an emotional state is detected, the pointer is placed on the first genre according to the order of priority in the table (as illustrated on FIG. 5). If we continue with the example of the emotional state corresponding to sadness, the first genre in the order of priority is the comic genre. It is therefore the comic genre that is pointed to (see the arrow on FIG. 5) at the time of the substep E61.

A substep E611 of extracting a list of at least one programme corresponding to the genre pointed to among the programmes in the catalogue is therefore performed by the audiovisual set-top box 10. Next, a substep E67 of displaying the extracted list comprising at least one audiovisual programme corresponding to the genre selected, i.e. to the genre pointed to in the table, is therefore performed. The display substep E67 corresponds to the display step E80 as described below.

At the substep E62, the audiovisual set-top box 10 detects whether the user is selecting a programme among the programmes in the extracted list corresponding to the genre pointed to in the priority table.

At the substep E68, the audiovisual set-top box 10 checks whether the user is selecting a programme from this extracted list of programmes corresponding to the genre pointed to. If the user selects a programme from this extracted list of programmes corresponding to the genre pointed to in the priority table, the set-top box passes to the substep E65.

If the user does not select a programme from this extracted list of programmes corresponding to the genre pointed to in the priority table, and if there are still genres to be offered in the priority table, the audiovisual set-top box 10 checks, at the substep E69, whether there are still genres to be offered in the priority table. If so, the audiovisual set-top box 10 passes to the substep E63 in which the pointer is shifted by a column in the priority table, and a substep E611 of extracting a new list of at least one programme corresponding to the genre pointed to and then a substep E67 of displaying a list of at least one audiovisual programme corresponding to the genre selected, i.e. to the genre pointed to in the table, are therefore performed. If no genre remains to be offered in the priority table, the audiovisual set-top box 10 points E64 to the default genre, and a substep E611′ of extracting a new list of at least one programme corresponding to the default genre and then a substep E67′ of displaying a list of at least one audiovisual programme corresponding to the default genre are implemented.

In the present example relating to the “sad” emotional state, if the user does not select a programme corresponding to the “comic” genre, then the pointer is shifted onto the “entertainment” genre, as illustrated on FIG. 7. A list of at least one audiovisual programme corresponding to the entertainment genre is then extracted and then displayed. If the user still does not select a programme in the extracted list of programmes corresponding to the entertainment genre, then the audiovisual set-top box 10 passes to the substep E69 and, if there are still genres to be offered in the priority table, the audiovisual set-top box 10 passes to the substep E63 so that the pointer is shifted to the following genre in the priority table (psychology genre), and so on as long as there are still genres to be offered in the priority table. Thus the audiovisual set-top box 10 checks, at the substep E69, whether there are still genres to be offered in the priority table. If so, the set-top box passes to the step E63 while if not the audiovisual set-top box 10 passes to the step E64. In this way, the pointer is shifted until the pointer reaches the default genre, which is classified last in the priority table for each emotional state (here the default genre associated with the sadness emotional state is the comic genre). The default genre is defined in advance. For example, it can be defined automatically by the audiovisual set-top box 10 or manually by the user when the audiovisual set-top box 10 is configured. In other words, the pointer runs through the priority table to successively offer the genres classified in the priority table corresponding to the emotional state detected. If no programme corresponding to these genres is selected by the user, a default genre is selected and a list of at least one programme associated with this default genre is extracted and offered.

In the case where the user selects a programme from the extracted list of programmes offered corresponding to a genre pointed to in the priority table, then the audiovisual set-top box 10 updates the priority table (MAJ substep E65) by incrementing the value of the counter of the number of readings for the genre corresponding to the programme selected for the emotional state detected, and the various genres belonging to the emotional state detected are reclassified so that:
priority 1 contains the genre having the most readings,
priority 2 contains the second genre having the most readings following the genre having priority 1; etc.

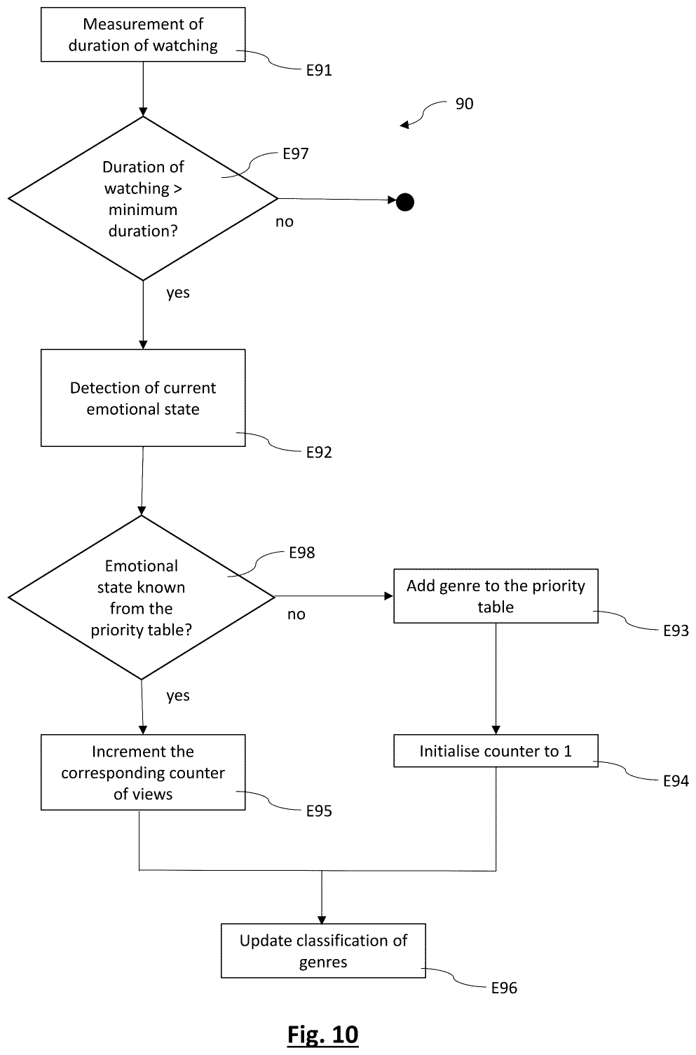

The step E80 of displaying a list of at least one audiovisual programme taking account of said sorting of said programmes in said catalogue comprises, in this example, the substeps of (illustrated on FIG. 9):
ordering E81 said at least one programme to be displayed, the ordering at least partly taking account ofa priority criterion of the at least one programme; and
displaying E82 the list of at least one programme according to the ordering. More precisely, when the step of implementing a sorting E60 of the programmes in the catalogue taking account at least of the emotional state detected and the genres associated with the emotional state detected is performed, a list of at least one programme corresponding to the genre pointed to is extracted. It is this extracted list that it is necessary to display E80 to the user. The ordering of the programmes in the extracted list takes account at least of a priority criterion associated with each programme. For example, the priority criterion can take account of the popularity of the programme with other users, of its date of release or making available, or of its commercial/marketing contribution (for example the advertising broadcast in some programmes is sold more expensively than in others, the producer of the programme may pay for it to have increased visibility, etc.). A combination of these data can be used to determine the priority criterion of each programme. Other data can obviously be used in determining this priority criterion. Preferably, the priority criterion is received in advance by the audiovisual set-top box 10, for example when information relating to the audiovisual programmes is received. In this example, the programmes in this extracted list are ordered according to their popularity with other users. Here the programmes offered are therefore ordered from the most popular programme to the least popular programme with the other users. For example, the popularity of the programme can be determined by taking account of the number of readings of the programme, of its rating by the other users, of its rating by the platforms, of its rating by the media or any other criterion. A combination of these criteria can also be used to determine the popularity of the programme.

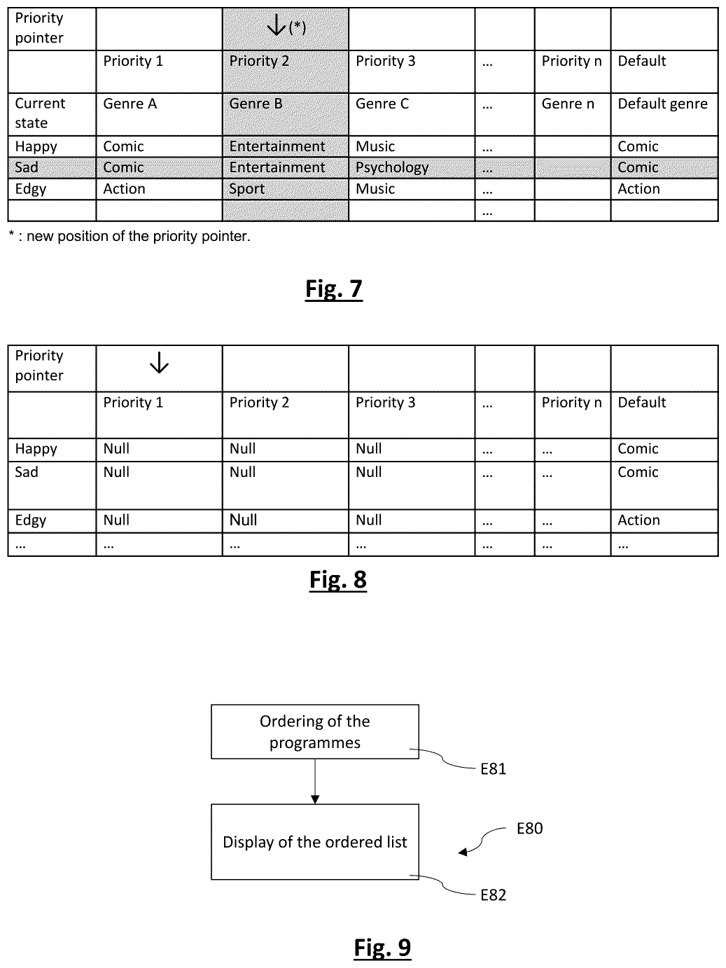

When the programmes in the list are put in order, the audiovisual set-top box 10, via the display means 13, displays the ordered list to the user to facilitate their selection. An example of a list of ordered programmes can consist of the presentation of the Top 10 comic films most watched (for the “sad” emotional state in priority 1) or of the Top 10 best ratedentertainment broadcasts (for the “sad” emotional state in priority 2), etc. FIG. 8 shows an empty priority table, as it can be when the audiovisual set-top box 10 is initialised. When the audiovisual set-top box 10 is initialised, the default column offering a default genre to each emotional state may however be given automatically.

The priority table can be filled in when it is initialised on the basis of a panel of users and by replicating the content of the panel of users in the content of the priority table. Another solution may consist of filling in the priority table by learning, i.e. gradually as the user selects a programme.

FIG. 10 illustrates an example of a method of filling in the priority table by learning. This filling-in method 90 comprises the steps of:
measuring, by the audiovisual set-top box 10 at the step E91, the duration of watching of a programme by the user;
checking, by the audiovisual set-top box 10 at the step E97, whether the duration of watching measured is greater than a predetermined minimum duration (for example ten minutes). If not, the audiovisual set-top box 10 interrupts the present algorithm. If so, the set-top box 10 passes to the step E92 of detecting the current emotional state of the user;

At the step E98, the audiovisual set-top box 10 checks whether the emotional state detected does not comprise the genre of the programme watched by the user in the priority table. If so, the audiovisual set-top box 10 passes to the step E93. If not, the audiovisual set-top box 10 passes to the step E95. At the step E93, the audiovisual set-top box 10 adds the genre of the programme watched by the user for the emotional state detected in the priority table and at the step E94 initialises the corresponding readings counter to 1;
if the emotional state detected already contains the genre of the programme watched by the user in the priority table, the audiovisual set-top box 10 increments E95 by one unit the value of the counter of readings of the genre corresponding to the emotional state detected;
the audiovisual set-top box 10 at the step E96 updates the classification of the genres by order of priority for the emotional state detected.

This method can be used in parallel with the recommendation method 100 described previously.

According to a second embodiment, the recommendation method 100 involves the current emotional state of the user detected to weight a predetermined score associated with a programme so as to make a recommendation of programmes to the user that takes account of their current emotional state.

In order to facilitate understanding of the method according to the second embodiment, in describing it account is taken only of the programme genres (and not of the subgenres). It will easily be understood that the method of the invention could take account, in addition to or in place of the genres, subgenres of the programmes without departing from the operating principle of this embodiment. One example embodiment taking account of subgenres is in particular described later in the description.

To do this, at the step E20 of associating the genres (and/or optionally the subgenres) with emotional states, the audiovisual set-top box 10 implements, as illustrated on FIG. 11, a substep of using E31 a counting table comprising a counter of readings by the user for each genre associated with an emotional state. More precisely, the counting table comprises a list of common emotional states with which offers of programme genres are associated. The counting table enables the audiovisual set-top box 10 to count E311, by incrementing by a value a counter of readings associated with each genre for each emotional state, the number of readings of a programme in each genre associated with each emotional state when the user selects a programme offered by the method of the invention. In other words, whenever a user selects and reads a programme that is offered to them by the method of the invention, the audiovisual set-top box 10 increments by a value the counter corresponding to the genre associated with the emotional state detected. In this way, the counting table makes it possible to count and store the number of readings of each genre associated with each emotional state.

It can also be envisaged counting the readings that are made outside the method of the invention. For example, it can be envisaged counting the readings made by a spontaneous selection of the user or when the user zaps from one programme to another, in particular. In order to optimise the relevance of the recommendations, the counting table can also be filled by learning, in a manner substantially similar to the technique described above in relation to FIG. 10.

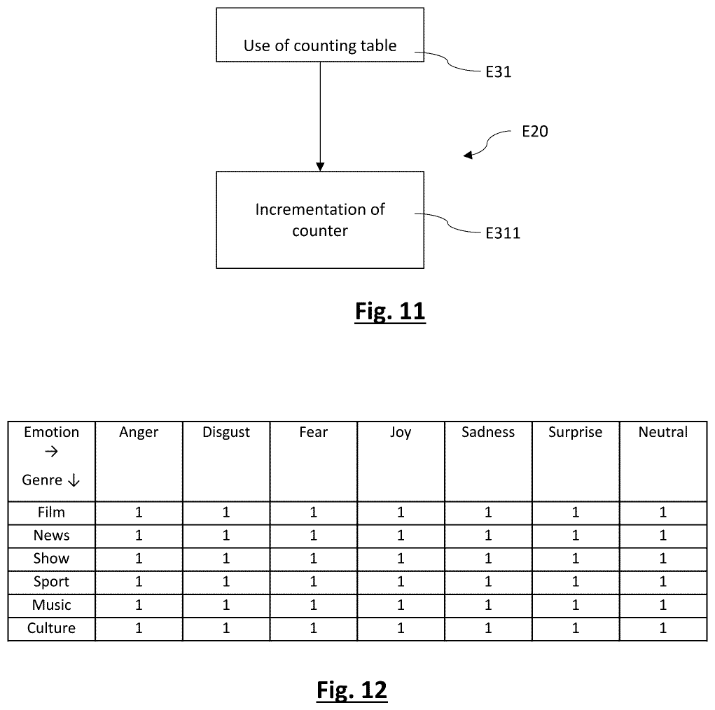

FIG. 12 shows a counting table when the audiovisual set-top box 10 is initialised. It can be seen that each counter is initialised with a value equal to 1. It will be understood that the counters could be initialised with any other value, preferably non-zero. The genres and emotional states given in the counting table can be fixed in a predetermined manner, or be recovered from an external server for example.

As before, the step E20 of associating the genres (and optionally the subgenres) with emotional states is followed by a step E40 of detecting a current emotional state of the user 12. This step E40 of detecting a current emotional state of the user 12 can for example use the known detection technique described in relation to the first embodiment and which consists of detecting the emotions of an individual from images or from the voice of the user 12.

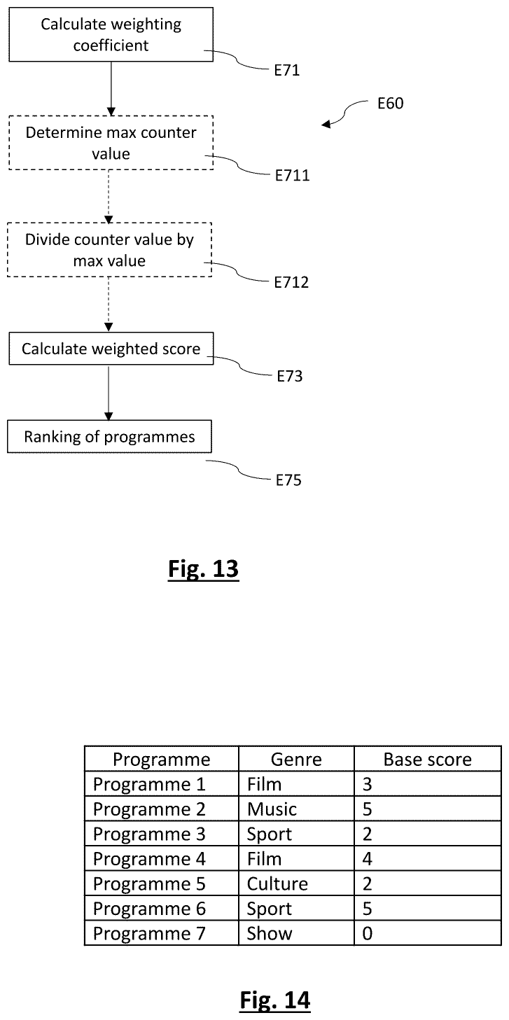

The step of making a sorting E60 of the programmes in the catalogue taking account at least of the emotional state detected and the genres associated with the emotional state detected comprises the substeps (illustrated on FIG. 13) of:
calculating E71, by the audiovisual set-top box 10, a weighting coefficient for each of the genres associated with the emotional state detected, the calculation of the weighting coefficient comprising:
   for the emotional state detected, determining E710, by the audiovisual set-top box 10, a maximum value from the counters associated with each of the genres;
   for each genre associated with the emotional state detected, dividing E712, by the audiovisual set-top box 10, the value of the corresponding counter by the maximum value determined;
for each programme included in a list of programmes offered in which each programme has a predetermined score and a genre, calculating E73, by the audiovisual set-top box 10, a weighted score by multiplying the predetermined score of the programme by the weighting coefficient corresponding to said genre;
ranking E75, by the audiovisual set-top box 10, said programmes in the list of programmes offered by decreasing order of weighted score.

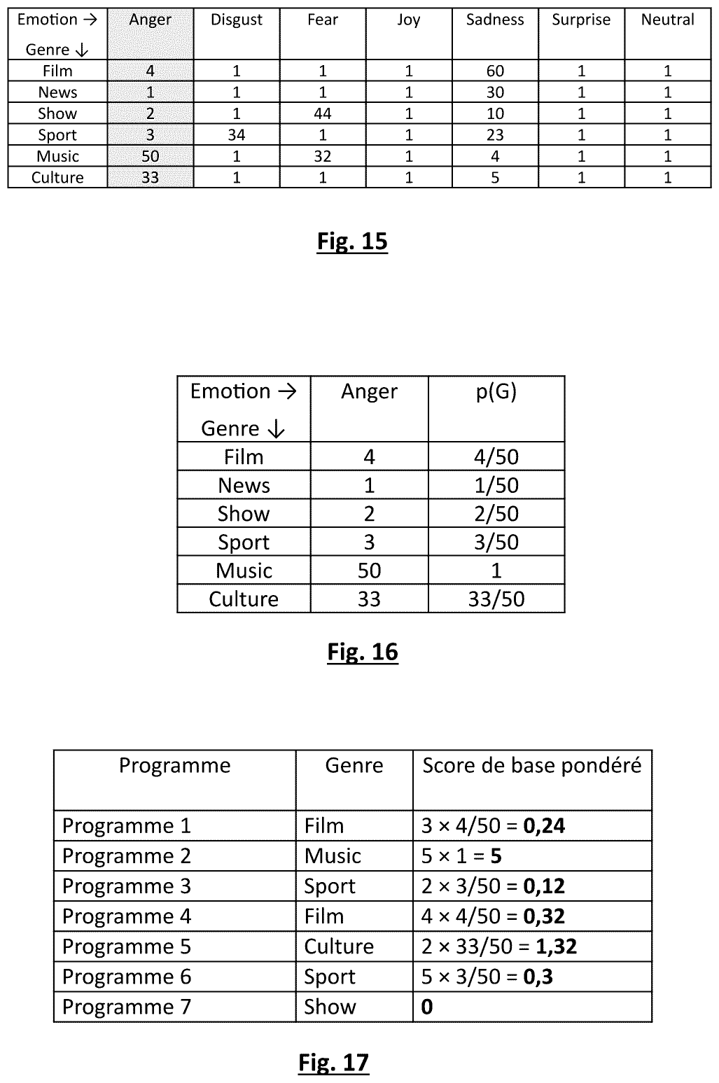

In this second embodiment, the audiovisual set-top box 10 receives a list of programmes in which a predetermined score is associated with each programme in the list (in addition to the bibliographical information and the indication of the genre (and optionally of the subgenre) conventionally received). For example, the predetermined score comprises a mark of between 0 and 5 corresponding to the popularity of the programme with the other users. The predetermined score can be determined by taking account of the number of readings of the programme (total or over a given period), of the age of the programme, of its mean mark given by the other users, of its rating by the platforms, of its rating by the media or of any other criteria. A combination of these criteria can also be used to determine the popularity of the programme. The predetermined score is independent of the user and is fixed by rules external to the method of the invention for example. FIG. 14 illustrates an example of a list of programmes in which a predetermined score is associated with each programme. In order to facilitate understanding of the step of making a sorting E60 of said programmes in said catalogue taking account at least of the emotional state detected and the genres associated with the emotional state detected, FIG. 15 illustrates an example of a counting table after several days of use. It will be noted that the counters of certain genres associated with certain emotional states have been incremented. In order, at the step E71, to calculate the weighting coefficient p(G), the audiovisual set-top box 10 at the step E711 determines first of all the maximum value among the counters associated with each of the genres. In other words, the audiovisual set-top box 10 will run through the counting table and seek which counter, for the current emotional state detected, is the highest. If we take as an example the emotional state corresponding to anger in the counting table in FIG. 15, the highest counting value corresponds to the music genre. The maximum value adopted is therefore that of the counter associated with the music genre for the emotional state of anger, namely a counter value equal to 50.

In a variant, the weighting coefficient could be calculated, or determined, differently, for example by dividing the value of the corresponding counter by the sum of the values of the counters associated with the emotional state detected, or by directly taking the value of the corresponding counter as the weighting coefficient.

Next, for each genre associated with the current emotional state detected, the audiovisual set-top box 10 will divide E712 the value of each counter by the maximum value determined previously.

In this example, and as illustrated on FIG. 16, the values of the counters of each genre associated with the emotional state of anger are divided by the maximum value determined, namely divided by 50. Thus the movie genre for the emotional state of anger has a weighting coefficient P(G) of 4/50, corresponding to the value of the counter of the movie genre for the emotional state of anger (i.e. a value of 4) divided by the maximum value determined (i.e. 50).

The weighting coefficient P(G) is in this example calculated for each genre corresponding to the emotional state detected.

When the weighting coefficients P(G) of each genre corresponding to the emotional state detected are calculated, the audiovisual set-top box 10 calculates E73 the weighted predetermined score of each programme in the list of programmes offered recovered by the audiovisual set-top box 10.

To do this, the audiovisual set-top box 10 multiplies the predetermined score of each programme by the weighting coefficient corresponding to the genre of the programme. If the example described here is referred to, the table illustrated on FIG. 17 shows the weighted score of each programme in the list of programmes in FIG. 14. It will thus be noted that the programme 1 has a weighted score of 0.24, corresponding to its predetermined score (namely 3) multiplied by the weighting coefficient of the film genre (namely 4/50).

The weighted score is calculated for each programme in the list of programmes offered to the audiovisual set-top box 10. The weighted score therefore makes it possible to weight the predetermined score of the programme by taking account of the current emotional state of the user.

Next, the programmes in the list of programmes offered to the audiovisual set-top box 10 are ranked E75 by decreasing order of weighted score so as to offer to the user a list of programmes that takes account of their current emotional state.

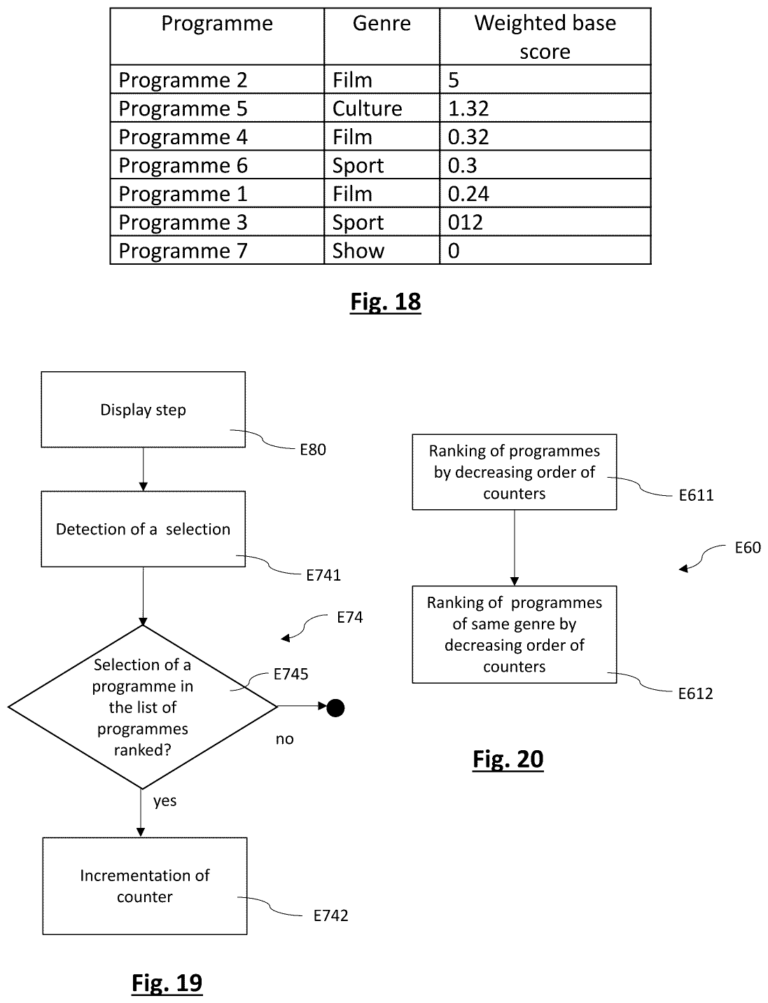

FIG. 18 therefore illustrates the list programmes ranked by decreasing order of weighted score. It will therefore be noted that, still for the emotional state of anger, the audiovisual set-top box 10 will offer to the user a programme of the music genre, then a programme of the culture genre, then a programme of the movie genre, etc

In this embodiment, the audiovisual set-top box 10, in sorting the programmes offered, relies on a weighting coefficient taking account of the detected emotional state of the user. The list of programmes displayed to the user can therefore comprise programmes of different genres and/or subgenres.

During the step E80 of displaying a list of at least one audiovisual programme taking account of the sorting of said programmes in the catalogue, the audiovisual set-top box 10 will display the list of ranked programmes described above.

The method 100 furthermore comprises, in this second embodiment, a step E74 performed by the decoder 10 of updating the counting table. To do this, and as illustrated on FIG. 19, following the step E80 of displaying the list of at least one audiovisual programme taking account of the sorting of the programmes in the catalogue, the audiovisual set-top box 10 will at the step E741 detect a selection of the user.

At the step E745, the audiovisual set-top box 10 checks whether the user has selected one of the programmes in the list of ranked programmes.

If not, the audiovisual set-top box 10 interrupts the present algorithm. If so, the audiovisual set-top box 10 updates the counting table by incrementing, at the step E742, by a value the counter corresponding to the genre of the programme selected for the current emotional state detected.

In a variant of this second embodiment, the audiovisual set-top box 10 does not calculate a weighted score at the step E60 of selecting at least one genre from at least the emotional state detected. According to this variant, and as illustrated on FIG. 20, the step of a sorting E60 of the programmes in the catalogue taking account at least of the emotional state detected and of the genres associated with the emotional state detected comprises the substeps of:
ranking E611, by the audiovisual set-top box 10, the programmes in the list of programmes offered, in which each programme has a predetermined score, by decreasing order of the counter associated with the genre and with the emotional state detected; then
ranking E612, by the audiovisual set-top box 10, the programmes in the list of programmes having the same genre offered by decreasing order of the predetermined score.

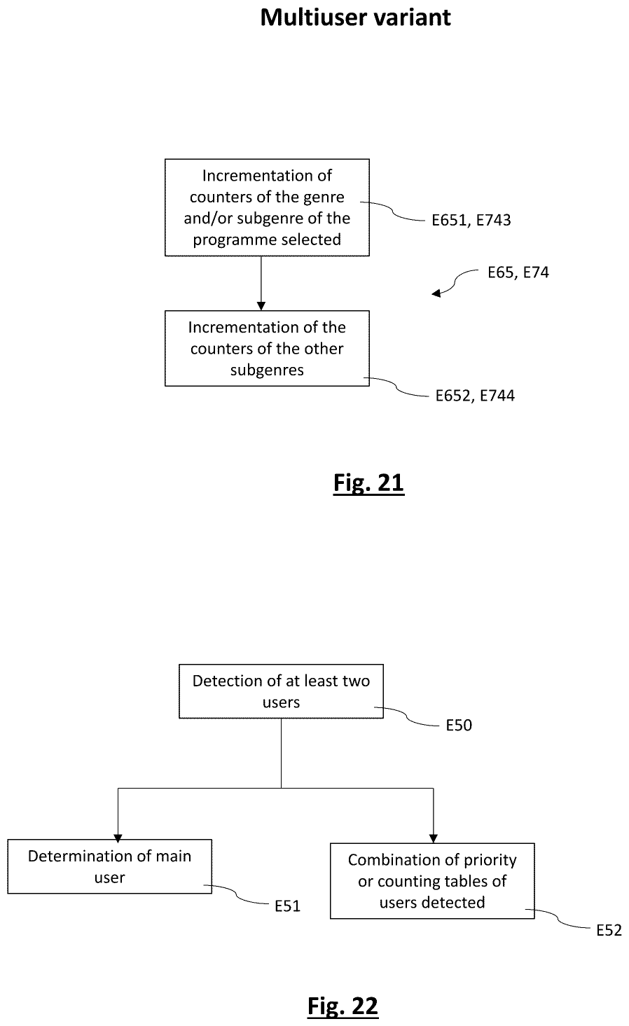

In another variant, compatible with the two embodiments of the invention described above as well as variants thereof, each genre comprises at least one subgenre. The priority and counting tables therefore comprise a counter for each of the subgenres, in addition to or instead of the counters associated with each genre. Thus, and as illustrated on FIG. 21, the step of updating E65 the priority table and the step of updating E74 the counting table each comprise the substeps of, when the at least one user watches one of the recommended programmes:
incrementing E651, E743 the counters of the genre and of the subgenre of the programme selected by the at least one user corresponding to the emotional state detected by a first value;
incrementing E652, E744 the counters of the other subgenres associated with the genre of the programme selected by the at least one user corresponding to the emotional state detected by a second value.

In this way, it is possible to take account of the genre and/or the subgenre of a programme in order to make an optimum recommendation to the user according to their current emotional state detected.

In an example that is not illustrated, the audiovisual set-top box 10 increments only the counters of subgenres. Thus the audiovisual set-top box 10 increments by a first value equal to two the value of the counter corresponding to the pair consisting of the main genre and the subgenre of the programme selected by the user, while the audiovisual set-top box 10 increments by a second value equal to one the value of the counters corresponding to the main genre but not to the subgenre of the programme selected.

In this example, if it is considered that the user selects a comic movie offered by the method, the comic movie is a programme belonging to the main genre “movie” and to the subgenre “movie/comedy”. Then the counter of the subgenre “movie/comedy” is incremented by a first value equal to 2, while the counters of the other subgenres of the “movie” genre (such as for example the subgenres “movie/drama”, “movie/science fiction”, “movie/adventure” etc) are incremented only by a second value equal to 1.

In the example described above, only the counters of the subgenres are considered. It will easily be understood that the number of readings of the genre can be obtained by incrementing the corresponding counter or by determining the value thereof from the sum of the counters of the subgenres that are associated therewith, for example.

It should be noted that several genres may have similar subgenres, for example there may be a “theatre” genre with subgenres similar to those of the “movie” genre of the previous example, such as “theatre/comedy”, “theatre/drama” etc. In a preferred embodiment of the invention, the subgenres of distinct genres are themselves considered to be distinct. Thus for example the “film/comedy” subgenre and the “theatre/comedy” subgenre each have a distinct counter. According to an alternative embodiment, similar subgenres use the same counter, so that there is one and the same counter common for the subgenres “film/comedy”, “theatre/comedy”, “serial/comedy” etc.

Thus the method according to the invention makes it possible to improve the targeting of the programmes recommended to the user.

It should be noted that the recommendation method 100 can be triggered in various ways. To do this, the method 100 can comprise a step E101 of launching/triggering said method 110:
when the audiovisual set-top box 10 is switched on; or
on the detection by the set-top box of the presence of at least one user by the audiovisual set-top box 10; or
on the detection by the set-top box of a selection/search for a programme by the at least one user.

It should be noted that, in the case where there are several users in the same home, each of the users can have their own priority and/or counting table. In this case, the recommendation method 100 can comprise a step of identification, by the audiovisual set-top box 10, of the user. For example, the identification of the user can be done manually via the control device 121 or automatically via the means 206 for detecting the emotional state. This is because the detection means 206 can be used, in addition to for detecting the current emotional state of the user, for making an identification of the user, for example by visual recognition or by audio recognition of the voice of the user. Techniques of the prior art make it possible to make such identification and are therefore not described in detail here.

It should be noted that, optionally, the recommendation method 100 can comprise a step of detecting E50, by the audiovisual set-top box 10, the number of users oriented towards the detection means 206. In this case, the recommendation method 100 can either take account of only one of the users, or take account of two or more users for making the recommendation.

To do this, and as illustrated on FIG. 22, in the case of detection E50 of at least two users, the recommendation method 100 furthermore comprises:
a substep of determining E51, by the audiovisual set-top box 10, the main user on the basis of at least one predetermined criterion, the substep of using E21 a priority table or the substep of using E31 a counting table being performed using said priority table or said counting table associated with said main user determined; or
a substep of combining E52, by the audiovisual set-top box 10, the priority or counting tables of each of the users detected, the substep of using E21 a priority table or E31 a counting table being performed using the priority table or the counting table resulting from this combination.

Thus, according to the first case, the audiovisual set-top box 10, at the step E51, determines a main user among the users detected so as to take account of only the priority or counting table of this main user. This substep E51 of determining the main user can for example rely on one or more of the following criteria: the age of the priority counting table, a highest counter of hours of watching programmes, a manual declaration by the users, etc.

In this first case, for implementing a sorting of the programmes in the catalogues, only the priority or counting table of the user determined as being the main user is taken into account. It can also be envisaged taking account of only the emotional state of the user determined as being the main user for making the recommendation.

In the second case, the audiovisual set-top box 10 combines E51 the priority or counting tables of each of the users detected. To do this, the audiovisual set-top box 10 can, for example, add the counters of the various users relating to each genre associated with each emotional state to obtain only one priority or counting table, referred to as the combined table. Thus this combined priority or counting table will count only one counter per given genre associated with a given emotional state, the value of this counter representing the sum of the counters of each user for the given genre associated with the given emotional state.

According to another approach (compatible with the first embodiment only), the audiovisual set-top box 10 can, for example, combine the priority tables of the various users by taking account of the counters of each of the users in one and the same priority table (and reorganizing the counters in the order of priority). Thus the preferences of each user can be taken into account, without distinguishing the secondary users with respect to a main user.

In both cases, it is possible to update the priority or counting tables of each of the users, either individually (for example when the table used is that of the main user) or collectively (i.e. the priority or counting table of each of the users can be updated according to the programme selected).

It is also possible, when it is wished to take account of the emotional state of each user, to mix the priority tables of the various users and to present one or more lists of programmes that take account of the emotional state of the various users.

According to another approach (compatible with the second embodiment only), the audiovisual set-top box 10 detects the current emotional state of each user present and calculates a weighting coefficient for each user from the current emotional state detected and from the counting table of each user. Next the audiovisual set-top box 10 calculates a weighted score for each programme by multiplying the predetermined score by the weighting coefficients of all the users present corresponding to the genre (and/or optionally to the subgenre) of the programme. Thus the recommendation also takes account of the emotional state of each user.

It should be noted that, when the audiovisual set-top box 10 is initialised, the creation of the priority and/or counting tables by the audiovisual set-top box 10 can take account of preferences of the user relating to the programme recommendation. For example, it is possible to offer a questionnaire to the user so as to take account of their preferences as long as a predetermined number of readings is not reached so that the recommendation method offers particularly relevant programmes based on the counters of each genre associated with each emotional state.

For example, the questionnaire offered by the audiovisual set-top box 10 can comprise questions of the following type:
“What genres of movies by priority do you like to look at when you are happy?”, so that the user is invited to select their first choice, then their second, and so on.
“Would you like to watch an entertainment programme when you are sad?”, so that the user is invited to reply yes or no.

According to another approach, when the audiovisual set-top box 10 is initialised, the creation of the priority and/or counting tables by the audiovisual set-top box 10 can take account of information coming from an external source, such as for example information supplied by the operator via the network 20.

Thus, instead of beginning with an empty priority table or a counting table in which all the counters are initialised with the value 1, the priority and counting tables can be prefilled, for example from an average of all the numbers of readings of a population of subscribers. Next, the learning process (i.e. updating the tables as the selections are made by the user) can continue as described previously in order to adapt the tables to each user.

The priority and/or counting tables on initialisation of the audiovisual set-top box 10 can be transmitted by the TCP/IP network or by a table or a private descriptor extending the DVB standard in an RF transmission.

Optionally, the priority and/or counting tables on initialisation of the audiovisual set-top box 10 can be linked to a general profile of the genre, example; “Movie profile”, “Sport profile”, which would be selected by the user when the set-top box is initialised. Optionally, the priority and/or counting tables on initialisation of the audiovisual set-top box 10 can be linked to a profile of the user relating to their age and/or sex, or any other criterion.

1. A method for recommending a list of at least one audiovisual programme from a catalogue of programmes to at least one user oriented to an audiovisual set-top box and display means, said programmes in said catalogue being categorised in genres, wherein the method comprises the steps performed by said audiovisual set-top box of:
associating said genres with emotional states;
detecting a current emotional state of said at least one user;
performing a sorting of said programmes in said catalogue taking account at least of said emotional state detected and of said genres associated with said emotional state detected;
displaying a list of at least one audiovisual programme taking account of said sorting of said programmes in said catalogue of programmes;
wherein said step of associating said genres with said emotional states comprises:
either a substep of using a priority table wherein, for each of said emotional states, said genres are classified in an order of priority, said classification taking account at least of a counter of readings of each genre associated with each emotional state, said priority table furthermore comprising a default genre attributed last according to the order of priority for each of said emotional states;
or a substep of using a counting table comprising a counter of readings by the user for each genre associated with an emotional state.

2. The method according to claim 1, wherein said step of implementing a sorting of said programmes in said catalogue taking account at least of said emotional state detected and said genres associated with said emotional state detected comprises a substep of:
pointing, in said priority table, to said genre classified first according to said order of priority for said emotional state detected, and
detecting a selection by the user and, if the user does not select a programme corresponding to said first genre pointed to, pointing to the following genre in the priority table until the user selects a programme in said genre pointed to or until said genre pointed to is said default genre.

3. The method according to claim 1, wherein said step of displaying said list of at least one audiovisual programme taking account of said sorting of said programmes in said catalogue comprises the substeps of:
ordering said at least one programme in said list of at least one programme to be displayed, said ordering at least partly taking account of a priority criterion of said at least one programme; and
displaying said list of at least one programme according to said ordering.

4. The method according to claim 1, wherein said step of performing a sorting of said programmes in said catalogue taking account at least of said emotional state detected and of said genres associated with said emotional state detected comprises the substeps of:
calculating a weighting coefficient for each of said genres associated with said emotional state detected;
for each programme included in a list of programmes offered in which each programme has a predetermined score and a genre, calculating a weighted score by multiplying said predetermined score of said programme by said weighting coefficient corresponding to said genre;
ranking said programmes in said list of programmes offered by decreasing order of weighted score.

5. The method according to claim 4, wherein the substep of calculating a weighting coefficient for each of said genres associated with said emotional state detected comprises:
for said emotional state detected, determining a maximum value from said counters associated with each of said genres;
for each genre associated with said emotional state detected, dividing said value of said corresponding counter by said maximum value determined.

6. The method according to claim 1, wherein said step of implementing a sorting of said programmes in said catalogue taking account at least of said emotional state detected and said genres associated with said emotional state detected comprises a substep of:
ranking the programmes in a list of programmes offered, wherein each programme has a predetermined score, by decreasing order of said counter associated with said genre and with said emotional state detected; then
ranking said programmes in said list of programmes having the same genre by decreasing order of the predetermined score.

7. The method according to claim 1, wherein it comprises a step of updating said priority table or said counting table, when said at least one user reads, during at least one predetermined period, one of said recommended programmes, by incrementing a value of said counter associated with said programme read by said at least one user.

8. The method according to claim 7, wherein each genre comprises at least one subgenre and wherein said priority table and said counting table comprise a counter of readings for each genre and a counter of readings for each subgenre, the step of updating said priority table and said counting table comprising the substeps of, when said at least one user reads one of said recommended programmes:
incrementing said counters of the genre and of the subgenre of the programme selected by said at least one user corresponding to said emotional state detected by a first value;
incrementing the counters of the other subgenres associated with the genre of the programme selected by said at least one user corresponding to said emotional state detected by a second value.

9. The method according to claim 1, wherein, when said audiovisual set-top box detects at least two users, the method comprises:
a step of determining the main user on the basis of at least one predetermined criterion, said substep of using a priority table or a counting table being performed using said priority table or said counting table associated with said main user determined; or
a step of combining said priority or counting tables of each of said users detected, said substep of using a priority table or a counting table being performed using the priority table or counting table resulting from this combination.

10. The method according to claim 1, further comprising a step of triggering said method:
when said audiovisual set-top box is switched on; or
when at least one user is detected by said audiovisual set-top box; or
when a programme is selected/sought by said at least one user.

11. An audiovisual set-top box for implementing the method for recommending a list of at least one audiovisual programme from a catalogue of programmes to at least one user according to claim 1, said programmes in said catalogue being categorised in genres, said audiovisual set-top box being intended to be connected to a remote database, a display means and a control device that can be used by a user for controlling said audiovisual set-top box, said set-top box comprising electronic circuitry adapted and configured for:
associating said genres with emotional states;
detecting a current emotional state of said at least one user;
performing a sorting of said programmes in said catalogue taking account at least of said emotional state detected of said genres associated with said emotional state detected;
said display means being intended to display a list of at least one audiovisual programme taking account of said sorting of said programmes in said catalogue;
wherein the association of said genres with said emotional states comprises:
either the use of a priority table wherein, for each of said emotional states, said genres are classified in an order of priority, said classification taking account at least of a counter of readings of each genre associated with each emotional state, said priority table furthermore comprising a default genre attributed last according to the order of priority for each of said emotional states;
or the use of a counting table comprising a counter of readings by the user for each genre associated with an emotional state.

12. A computer program product comprising instructions for implementing, by an audiovisual set-top box, the method according to claim 1, when said program is executed by a processor of the audiovisual set-top box.

13. A storage medium storing a computer program comprising instructions for implementing, by an audiovisual set-top box, the method according to claim 1, when said program is executed by a processor of the audiovisual set-top box.
